(19) United States
(12) Patent Application Publication
(10) Pub. No.: US 2026/0306559 A1
(43) Pub. Date: Oct. 8, 2026

(54) FORMULATION OF A PROBIOTIC STABLE AT BAKING TEMPERATURES AND ENCAPSULATION OF THE SAME TO ENSURE ITS LIFE ON SHELF FOODS UNTIL ITS VIABILITY IN CONSUMPTION

(71) Applicant: GRUPO BIMBO, S.A.B. DE C.V., Mexico City (MX)

(72) Inventors: Rocio Alejandra Chavez Santoscoy, Mexico City (MX); Sergio Serna Othon Saldivar, Mexico City (MX)

(73) Assignee: GRUPO BIMBO, S.A.B. DE C.V., Mexico City (MX)

(21) Appl. No.: 19/481,230

(22) PCT Filed: Jun. 6, 2023
(86) PCT No.: PCT/MX2023/050031

§ 371 Date: Nov. 4, 2025

(30) Foreign Application Priority Data
May 4, 2023 (MX) MX/A/2023/005340

(51) Int. Cl. A61K 35/742; A21D 8/04; A61K 9/16; A61K 35/00; A61K 35/747

(52) U.S. Cl. CPC A61K 35/742; A21D 8/04; A61K 9/1652; A61K 9/1658; A61K 35/747; A61K 2035/115

(57) ABSTRACT

A scalable and affordable package is provided that allows probiotics to survive baking processes, minimizing as much as possible the over-addition necessary to achieve the CFU necessary to achieve the desired levels of probiotics. A process of forming stabilized probiotic package for a liquid-based food product includes preparing core pellets containing probiotics and substrate. The core pellets are overcoated.

### FIELD OF INVENTION

The global market for functional foods is expanding every day, due to the growing demand from consumers due to an increased awareness of consumers to include healthy foods that offer health benefits in their daily diet.

The market for functional products worldwide is worth 139.2 billion dollars, which for Mexico is equivalent to 5% of the world value, with a constant annual growth rate of 3.9%. The probiotics market worldwide represents 52.9 billion dollars, in Mexico, it is worth 0.04 billion dollars, with a growth rate of 7% per year.

However, North America, especially the United States, has had an accelerated growth in this market where 3% of new food and beverage products contain probiotics.

### BACKGROUND TO THE INVENTION

Probiotics are often found in dairy products, but they are increasingly being incorporated into other foods, such as juices, cereals, bars, etc. Most probiotics are sensitive to heat, so their survival during thermal processes is a major obstacle. The heat generated in baking processes can lead to significant losses during the bread process and storage. In addition, probiotics must survive and grow inside the host to exert their beneficial effects on health. They must be metabolically stable and active, survive acidic stomach conditions, and reach the gut in optimal amounts. Similarly, factors such as pH, presence of oxygen, temperature and humidity in the product must be ideal so as not to affect its growth and survival.

The encapsulation of nutraceuticals, including probiotics, is a strategy that allows a functional ingredient to be given stability under specific conditions. This technology solves some problems in the process, because it captures the main ingredient as the central core and is coated with an inert or protective layer that improves stability during passage through the gastrointestinal tract. In the case of probiotics for use in bakery products, it is necessary to preserve the viability of the probiotics at baking temperatures, it is also advisable to stabilize them so that they can reach the human intestine viable, where they can exert their beneficial action on health.

Currently, encapsulation is an important alternative for the implementation of probiotics in food, to allow them to arrive alive at the site where they are going to exert their action, protecting them from environmental conditions (heat, light, air, humidity)

There are different methods of encapsulation of bioactive compounds such as emulsion, liposome formation, electrospinging, and spray driying. Most of them involve the use of solvents or technologies that are difficult to scale in the industry (Ayala-Fuentes et al., 2021). However, spray drying is a scalable technique at an industrial level, fast, with low operating costs and that does not require the use of solvents and does not generate pollutants, therefore, it is defined as sustainable

Patent Application MX/a/2016/006536; Production process and formulation of acidified fermented beverages based on strains with probiotic activity of Lactobacillus paracasei tolerans pfth1-10 and pfth1-22, from agave atrovirens juices and honeys. This document describes the process of transformation of honeys or syrups and agave saps from two different bacterial cultures belonging to the species Lactobacillus paracasei tolerans. The aforementioned microorganisms have in vitro probiotic activity, as they produce bacteriocins and organic acids produced in the fermentation of honey and agave juices; which results in the inhibition of the growth of pathogenic and toxigenic enterobacteriaceae. During fermentation, the strains of bacteria that are the subject of the invention rapidly consume the monosaccharides and some oligosaccharides that are available, while secreting pleasant flavors and aromas. At the end of a fermentation process of between 48 and 120 h; and a period of stabilization of the product at refrigeration temperature for 2 to 21 days, an acidulated drink is obtained that contains the probiotic microorganisms themselves, as well as polymerized oligofructans and fructans. This product does not contain alcohol, has a very low content of glycemic sugars, and is free of animal fats, cholesterol, or allergens derived from milk proteins. It is therefore a functional drink, which contains probiotic microorganisms; prebiotic oligosaccharides; and short-chain organic acids.

Unlike the present invention in this document does not speak of high temperatures to preserve the properties of a probiotic, the process of transformation of honeys or syrups and saps of agave from two different bacterial cultures belonging to the species lactobacillus is not extrapolated in any way to the process we have because this is fermentation and has nothing to do with heat, or drying of bacteria.

International Application MX/a/2014/012235; Use of lactic acid bacteria to prepare fermented food products with greater natural sweetness.

This document describes the aim of providing an alternative to adding sweeteners to fermented milk products to achieve the desired sweet taste without added calories. In addition, it would be very advantageous to establish a method of reducing lactose in fermented milk products to a level that is acceptable to consumers who are lactose intolerant. The above problems were solved by providing the mutant strains of Streptococcus therrrophilus and the mutant strains of Lactobacillus delbrueckii subspecies bulgaricus that excrete glucose into milk when milk is inoculated and fermented with such strains of Streptococcus thermophilus and strains of Lactobacillus delbrueckii subspecies bulgaricus. Thus, the present invention relates to strains of Streptococcus thermophilus and Lactobacillus delbrueckii subspecies bulgaricus that secrete glucose into the milk substrate during fermentation, as well as to mixed cultures comprising the strains of Streptococcus thermophilus and the strains of Lactobacillus delbrueckii subspecies bulgaricus, to starter cultures comprising the strains and to dairy products made from the cultures. This method also refers to the use of strains to decrease the lactose content of a fermented food product and to support the growth of BB-12 (r) probiotics.

This document does not talk about high temperatures to preserve the properties of a probiotic, likewise this is a fermentative process, in our process there is no fermentation of basteries and fermented products are not used for their desired sweet flavor without the added calories and instead this technology does not affect the knowledge of the final product.

Publication: KR2021181788A; Title: A non-stop production process to improve freeze-drying survival, heat tolerance, storage stability and digestive stability of probiotics using spontaneous matrix encapsulation technique.

This paper describes a method for preparing alginate-hydrogel-encapsulated probiotics that involves (a) growing probiotics in a medium containing alginic acid salts and salts to form hydrogels by combining with alginic acid to spontaneously generate alginate-hydrogels simultaneously with probiotic proliferation, and (b) recovering the probiotics encapsulated by the spontaneously produced alginate hydrogel. Encapsulated probiotics are encapsulated in an alginate hydrogel, encapsulated probiotics are matrix type. The medium of step (a) also comprises a potentiating encapsulation where the encapsulation enhancer is chosen from starch, crystalline cellulose, chitosan, carboxymethylcellulose (CMC) and skim milk powder. The probiotics are Lactobacillus sp., Bifidobacterium sp., Streptococcus sp., Lactococcus sp., Enterococcus sp., Leuconostoc sp., Pediococcus sp. and Weissella sp.

Composition: The salt of alginic acid is 0.1-40 g/1, and the salt to form a hydrogel by binding with alginic acid is contained in the medium at 0.5-10 g/l. The encapsulation enhancer is contained in the medium in an amount of 1-20 g/1. Preferred Method: Step (a) involves dissociating the cation from the salt that binds to alginic acid to form a hydrogel by the acid generated by the culture of the probiotics, and spontaneous binding of the dissociated cation and alginic acid. The method further involves (c) freeze-drying the encapsulated probiotics with the recovered hydrogel alginate.

This paper in particular mentions an encapsulation where the encapsulation enhancer is chosen from starch, which may be relevant to a thermostable stabilized probiotic.

This particular document does mention temperatures and their tolerance to them. But in the present invention a hydrogel composition with alginate and a complex carbohydrate is not used. In the present invention a hybrid solid carbohydrate-protein nanoparticle is used, the formulation is different in the type of encapsulant and therefore its application, ours is for use in food and all its ingredients are for use in food and especially in bakery products.

Publication: KR2006130968A; Title: Probiotic Bifidobacterium Boum R5 strain with excellent acid tolerance, bile acid tolerance, oxygen tolerance and heat stability and probiotic composition comprising the same or your medium culture.

This document describes a Bifidobacterium boum R5 probiotic strain and a probiotic composition comprising the same strain are provided to inhibit the growth of various intestinal pathogenic bacteria effectively, and improve acid tolerance, bile acid tolerance, oxygen tolerance and heat stability of the strain.

The probiotic Bifidobacterium boum R5 strain (KCCM 10669P) capable of inhibiting gut growth provides pathogenic bacteria, where the Bifidobacterium boum R5 strain (KCCM 10669P) is isolated from the contents in the first stomach of a cow; and the intestinal pathogen bacteria is Salmonella gallinarum, Salmonella enteritidis, Salmonella typhimurium, Staphylococcus aureus, enterotoxigenic Escherichia coli, Listeria monocytogenes, Campylobacter jejuni or Clostridium perfringens. The probiotic has a composition comprising the probiotic Bifidobacterium boumR5 strain (KCCM 10669P) and its culture medium, where the composition is used as food additives. The composition is heat-dried or freeze-dried, and the amount of probiotic Bifidobacterium boumR5 (KCCM10669P) strain is 105 to 1012 cfu/g based on the total weight of the composition.

This document deals with an isolated and modified strain, but in the present invention no strain is used and we do not modify the strains themselves. It is a coating material.

Publication: KR201696641A, title: Lactobacillus ferment p19119 with biofunctional activities and high heat stability as a probiotic without antibiotic resistance.

This paper describes a new strain of Lactobacillus fermentum PL9119 used in composition for intestinal disorders, and is used in healthy and functional foods such as milk, yogurt, soy milk, meat products, sausages, bread, beverages, capsules or stick packs. The Lactobacillus fermentum PL9119 strain comprises a nucleotide sequence of 16S rRNA that has 648 nucleobases, given in the specification, where Lactobacillus fermentum strain PL9119 is deposited in Korean Collection for Type Cultures (KCTC 13038BP). The strain is selected from the group formed by the strain resistant to ampicillin, gentamicin, erythromycin, clindamycin, tetracycline, chloramphenicol, vancomycin, synercid, quinupristin, dalfopristin or a combination, where the antibiotic agent is not resistant to the group of linezolid or rifampicin that is free from the risk of resistance metastasis. The strain or culture solution in the prophylactic composition is excellent in acid resistance and bile resistance. The strain is selected from the group consisting of the O157:H7 strain of Escherichia coli preserved in the American Type Culture Collection (ATCC No: 43894), Salmonella typhimurium conserved at the Canadian Centre for Agri-Food Research in Health and Medicine (CCARM No: 8001), Salmonella enteritidis preserved at the Canadian Centre for Agri-Food Research in Health and Medicine (CCARM No. 8010), Enterococcus fecalis preserved in the American Type Culture Collection (ATCC No: 29212), Staphylococcus aureus preserved in the Canadian Centre for Agrifood Research in Health and Medicine (CCARM No: 0045), and Listeria monocytogenes preserved in the American Type Culture Collection (ATCC No: 19113C3a). Functional food for health promotes gut health activity through food inhibition, bacterial growth poisoning, and lipase production.

Unlike what is described, the present invention does not use that strain and neither are the strains modified by themselves, it is a coating material.

Publication: BR1120133284A; Title: Procedure for the preparation of heat and moisture resistant probiotic bacteria in the form of stabilized probiotic granules for a liquid-based food product, process for the preparation of a liquid-based food product, stabilized probiotic granules for mixing in a liquid base food product, and, a food product.

Thermally processed or thermally processable healthy food products that beneficially affect the consumer's gut microbial balance are provided herein.

Food products are particularly liquid-based products that comprise a probiotic component capable of resisting heat and humidity.

The process (I) for preparing heat and moisture resistant probiotic bacteria in the form of stabilized probiotic pellets for a liquid-based food product comprises: (i) preparing core pellets containing probiotic bacteria, substrate, and optionally other food-grade ingredients; (ii) optionally, coat the core granules with an inner layer to obtain sealed core granules (A); (iii) optionally coat (A) with an outer layer comprising a thermosensitive gel-forming polymer to obtain protected core granules (B); and optionally (iv) coating (B) per outer layer comprising a water-soluble polymer.

The granules comprise a core of probiotic bacteria on a substrate or mixed with a substrate, and at least one inner layer, one outer layer, or one outer layer comprising a water-soluble polymer or an erodible polymer. The substrate comprises; a component comprising bacterial supplement, stabilizer, filler and/or binder; or a prebiotic saccharide.

This document is about a coating gel, the formation process is different because it is liquid-based, the size of the final result is different from ours reaches up to 4 microns only.

### BRIEF DESCRIPTION OF THE DRAWINGS

FIG. 1 shows the spray drying scheme. The main parts that make up a spray drying equipment are a container where the solution to be dried by spray is located, In the drying chamber are the atomizing nozzle where the solution is sprayed, very close to this nozzle there is an outlet of hot air that will dry the small droplets formed from the solution, Finally, in the drying chamber at the end, the dry product is obtained, usually with a particle diameter similar to the opening of the atomizing nozzle.

FIG. 2, shows the morphology of probiotics used. On the left is Bacillus clausii, a sporulated and circular microorganism smaller than 1 micrometer, which is very small in size compared to the second microorganism used. On the right is Lactobacillus acidophilus, the second a microorganism used, which is significantly larger, has the shape of a bacillus and an average size of 2 micrometers

FIG. 3 shows the encapsulation optimization of Bacillus clausii. The optimal point was determined at 35% Inulin, 60% pea protein, 5% maltodextrin and an initial concentration of probiotics in the order of 4×10¹¹ CFU.

FIG. 4 shows the encapsulation optimization of Lactobacillus acidophilus. The optimal point was determined to be 30% Inulin, 40% pea protein, 25% maltodextrin and an initial concentration of probiotics in the order of 4×10⁸ CFU.

FIG. 5. Optimal formulations for probiotics These formulations were obtained through 2 experimental designs with spray separation technology. The integration of the two experimental designs served as a basis to achieve an optimal formulation to propose, a formulation for the combination of both probiotics in a single microencapsulate.

FIG. 6 shows images of the morphology of the microencapsulates obtained by scanning electron microscopy (SEM). Microencapsulated scanning electron microscopy (SEM) obtained. a) The encapsulation of Basillus clausii is observed 3.53×10¹⁰ CFU/g of encapsulation, b) The encapsulation of Lactobacillus acidophilus 1.22×10⁸ CFU/g of encapsulation is observed, and in c) the encapsulation of 2 probiotics containing Lactobacillus acidophilus in 6.69×10⁸ CFU/g of encapsulation and Basillus clausii in 2.24×10¹⁰ CFU/g of encapsulation.

FIG. 7 shows the scaling of obtaining microencapsulates. The process was scaled from 500 mL of solution, where 4 to 5 were obtained, to a process of 5 L, where approximately 150 g per run of each microencapsulate was obtained.

FIG. 8, the micro-opanification prototypes with packages generated by direct method are shown. From left to right you can see the control bread, the bread with microencapsulation from Spain (positive control), encapsulated with L. acidophilus, encapsulated with B. clausii and the encapsulated with the 2 probiotics (L. acidophilus and B. clausii).

FIG. 9. 460 g baking prototypes with encapsulations generated by sponge method. From left to right, bread with the 2 probiotics (L. acidophilus and B. clausii), bread with microencapsulated B. clausii, bread with microencapsulated L. acidophilus and finally control bread can be seen.

FIG. 10 describes the in vitro digestion process.

### DETAILED DESCRIPTION OF PREFERRED MODALITIES

The encapsulation of nutraceuticals, including probiotics, is a strategy that allows a functional ingredient to be given stability under specific conditions. This technology solves some problems in the process, because it captures the main ingredient as the central core and is coated with an inert or protective layer that improves stability during passage through the gastrointestinal tract (Vasishtha, 2005). In the case of probiotics for use in bakery products, it is necessary to preserve the viability of the probiotics at baking temperatures, it is also advisable to stabilize them so that they can reach the human intestine viable, where they can exert their beneficial action on health. Currently, encapsulation is an important alternative for the implementation of probiotics in food, to allow them to arrive alive at the site where they are going to exert their action, protecting them from environmental conditions.

There are different methods of encapsulation of bioactive compounds such as emulsion, liposome formation, electrospinging, and spray driying. Most of them involve the use of solvents or technologies that are difficult to scale in the industry (Ayala-Fuentes et al., 2021). However, spray drying is a scalable technique at an industrial level, fast, with low operating costs and that does not require the use of solvents and does not generate pollutants, therefore, it is defined as sustainable.

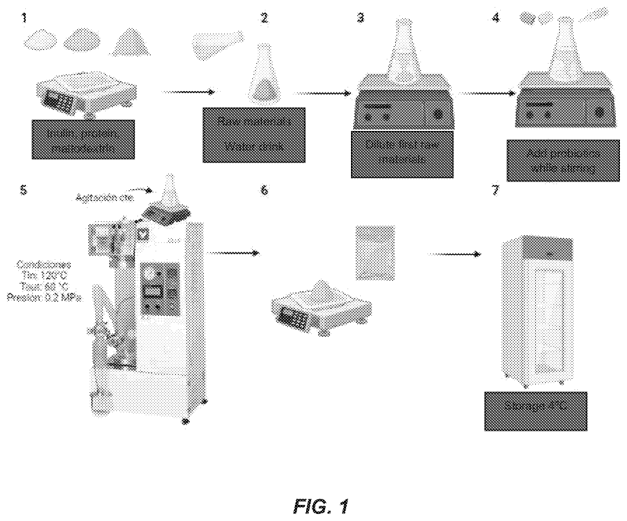

Spray drying is a technology widely used in the food industry, to produce powdered ingredients, such as soluble coffee, baby milk formulas, among others. The advantages of spray drying is that it is a very robust, scalable technique and very good yields are obtained. FIG. 1 presents a diagram explaining the main components of the technology.

The main objective was to develop a scalable and affordable encapsulation process that allows the probiotics to survive our baking processes, minimizing as much as possible the over-addition necessary to achieve the CFU necessary to make the corresponding declaration.

The following stages were contemplated to carry out the process:
1) Development of Functional Ingredient. Generate an experimental Box-Behnken design, because it reduces the number of runs to optimize the formulation. that helps us validate the microencapsulation of probiotics already reported, to preserve viability at high temperatures (170°−210° C.). 24 runs of experiments must be performed to complete the experimental design and optimize the response variables: Size, viability, and loading efficiency.
2) Generation of a baking prototype by Microbreading. To evaluate the behavior of the optimized formulation in baking, performing 16 experiments that allow us to determine the necessary dose to ensure the desired concentration of probiotics. Finally, it is contemplated to make a commercial-sized bread for sensory analysis in the company.
3) Life evaluation of developed bread shelf. Test 3 shelf life conditions, considering 3 temperatures 4° C., 25° C. and 40° C., at a relative humidity (60%) to quantify the shelf life of the final product and the viability of the probiotics in CFU/g.
4) In vitro digestion evaluation of the developed bread. Assess the number of viable microorganisms by performing an in vitro digestion assay to determine the number of viable microorganisms that would potentially reach the human intestine.

### Stage 1: Development of Functional Ingredient.

It was proposed to generate an experimental design (Box-Behnken) that is a spherical and rotating response surface, which includes a central point and midpoints between the corners, circumscribed on a sphere, because it reduces the number of runs to optimize the formulation. that helps us validate the microencapsulation of probiotics already reported, to preserve viability at high temperatures (170°−210° C.). 24 runs of experiments must be performed to complete the experimental design and optimize the response variables: Size, viability, and loading efficiency.

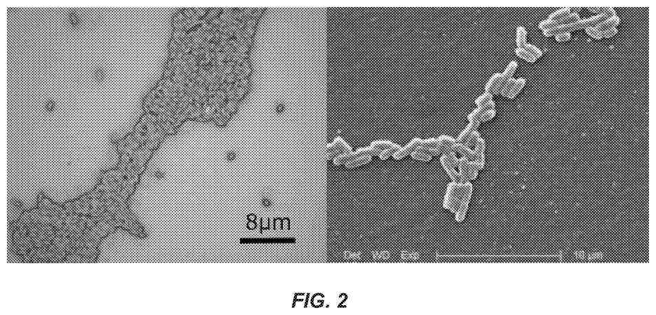

First, 3 proofs of concept were carried out, to determine that through spray drying it was possible to obtain an encapsulation of 2 probiotics with different morphological characteristics (FIG. 2).

These strains were chosen for their functional properties. Lactobacillus acidophilus has been widely reported because it maintains and restores the health of the microbiota in the digestive system, supports the immune system, inhibiting different pathogenic bacteria. As for Bacillus clausii, its properties, in addition to maintaining and restoring the microbiota of the digestive system and supporting the immune system, have been reported to maintain intestinal permeability, and therefore improve the absorption of nutrients, also reduce the sensation of bloating, flatulence, diarrhea or constipation and relieve gastrointestinal pain and inflammation.

Subsequently, 2 experimental designs were carried out for the optimization of 2 probiotics, a sporulate —Bacillus clausii— and a lactobacillus—Lactobacillus acidophilus—. In these experimental designs, the concentration of inulin and the vegetable protein used were varied, and the response variable was the viability of the organisms (Table 1 and Table 2).

TABLE 1
Experimental design performed for Bacillus clausii*.

| | Factor 1 | Factor 2 | Factor 3 | |
|---|---|---|---|---|
| | A: Inulin | B: pea protein | C: CFU | Spray Drier |
| Run | % | % | billions | CFU/g |
| 1 | 30 | 40 | 3 × 10¹² | 2.58 × 10⁸ |
| 2 | 25 | 60 | 2 × 10¹² | 1.00 × 10⁷ |
| 3 | 30 | 40 | 3 × 10¹² | 1.55 × 10⁹ |
| 4 | 30 | 40 | 3 × 10¹² | 5.03 × 10⁸ |
| 5 | 25 | 20 | 4 × 10¹² | 4.75 × 10⁹ |
| 6 | 35 | 20 | 2 × 10¹² | 1.00 × 10⁹ |
| 7 | 30 | 40 | 3 × 10¹² | 7.00 × 10⁹ |
| 8 | 30 | 40 | 3 × 10¹² | 2.85 × 10⁹ |
| 9 | 35 | 60 | 4 × 10¹² | 3.25 × 10¹¹ |

*The factors that varied were A: % of Inulin, and B: % of pea protein, the response variable was the viability obtained at the end of spray drying in Colony Forming Units per gram (CFU/g)

TABLE 2
Experimental design performed for Lactobacillus acidophilus*

| | Factor 1 | Factor 2 | Factor 3 | |
|---|---|---|---|---|
| | A: Inulin | B: pea protein | C: CFU | Spray Drier |
| Run | % | % | ×10\hat{ }8 | CFU/g |
| 1 | 25 | 60 | 4.7 × 10⁸ | 2.5 × 10⁷ |
| 2 | 30 | 40 | 7.05 × 10⁸ | 1.84 × 10⁷ |
| 3 | 25 | 20 | 9.4 × 10⁸ | 2.00 × 10⁷ |
| 4 | 30 | 40 | 7.05 × 10⁸ | 1.05 × 10⁸ |
| 5 | 35 | 20 | 4.7 × 10⁸ | 7.57 × 10⁷ |
| 6 | 35 | 60 | 9.4 × 10⁸ | 2.00 × 10⁷ |
| 7 | 30 | 40 | 7.05 × 10⁸ | 1.75 × 10⁷ |
| 8 | 30 | 40 | 7.05 × 10⁸ | 2.29 × 10⁷ |
| 9 | 30 | 40 | 7.05 × 10⁸ | 1.76 × 10⁷ |

*The factors that varied were A: % of Inulin, and B: % of pea protein, the response variable was the viability obtained at the end of spray drying in Colony Forming Units per gram (CFU/g)

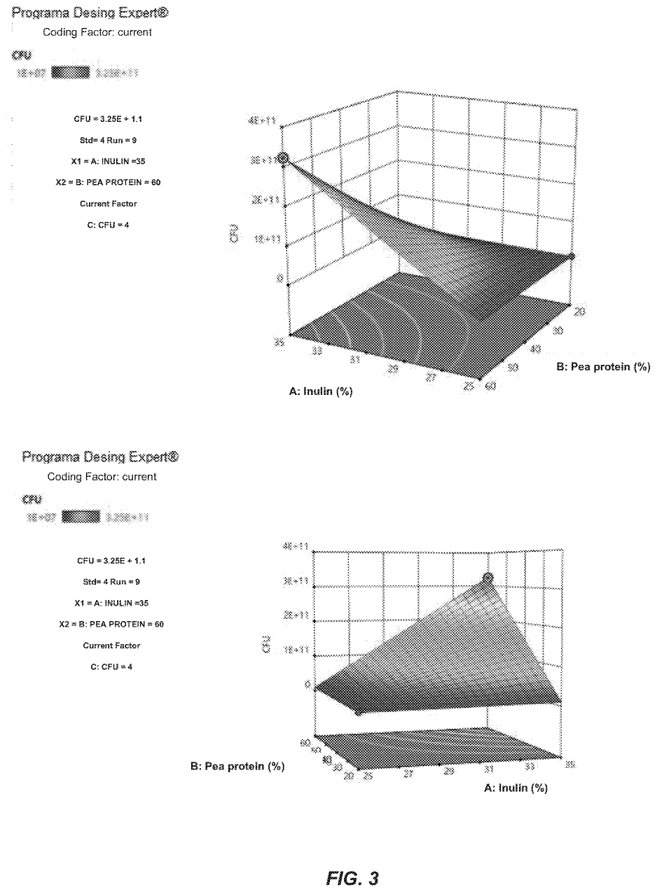

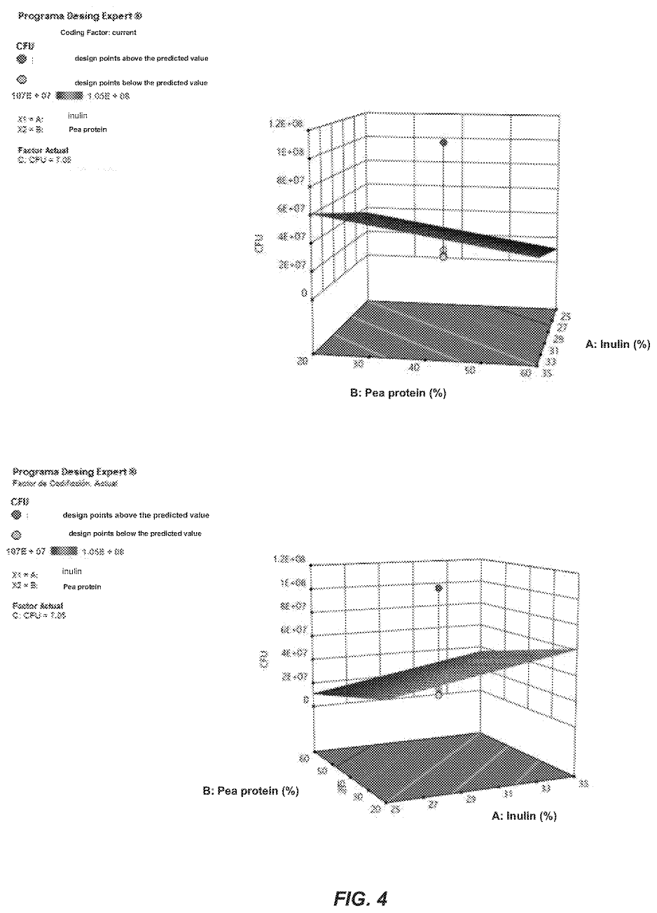

The optimization of both experimental designs was carried out through a surface analysis in the Design-Expert® software. The optimal conditions for both experimental designs were obtained (FIG. 3 and FIG. 4). Under these conditions, the optimal formulation for both probiotics was established.

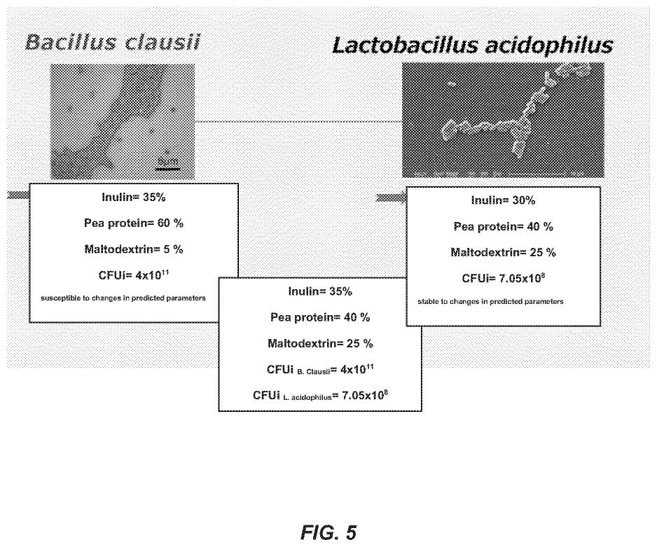

FIG. 3. Optimization of Bacillus clausii encapsulation. The optimal point was determined at 35% Inulin, 60% protein, in this way the optimal formulation parameters for each of the microorganisms were obtained and a formulation was proposed for the encapsulation of both probiotics in a single microencapsulated ingredient obtained by spray dryer (FIG. 5).

In this stage, 3 encapsulates with temperature stability characteristics were obtained, whose concentration of microorganisms is:
1. Basillus clausii 3.53×10¹⁰ CFU/g potting
2. Lactobacillus acidophilus 1.22×10⁸ CFU/g potting
3. Encapsulated with 2 probiotics, it has Lactobacillus acidophilus in 6.69×10⁸ CFU/g of encapsulation and Basillus clausii in 2.24×10¹⁰ CFU/g of encapsulation.

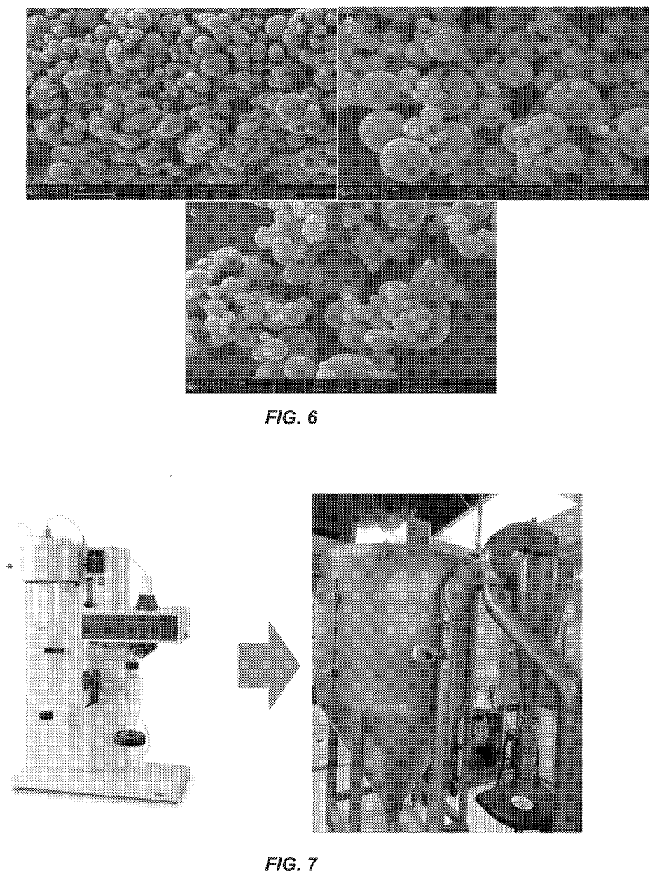

FIG. 6. Microencapsulated scanning electron microscopy (SEM) obtained. a) The encapsulation of Basillus clausii is observed 3.53×10¹⁰ CFU/g of encapsulation, b) The encapsulation of Lactobacillus acidophilus is observed at 1.22×10⁸ CFU/g of encapsulation, and in c) the encapsulation of 2 probiotics containing Lactobacillus acidophilus in 6.69×10⁸ CFU/g of encapsulation and Basillus clausii in 2.24×10¹⁰ CFU/g of encapsulation.

The obtaining of these encapsulates was scaled from a team that obtained 4 to 5 grams per run (laboratory scale) to one where approximately 150 g per run was obtained (pilot). With the same characteristics (FIG. 7). This shows that the formulation is robust and that it can be scaled up to obtain a similar viability.

### Stage 2: Generation of a Bakery Prototype by Microbreading.

In this stage, the aim was to evaluate the behavior of the optimized formulation in baking, carrying out 16 experiments that allowed us to determine the necessary dose to ensure the desired concentration of probiotics. Finally, it was contemplated to make a commercial-sized bread for sensory analysis in the company.

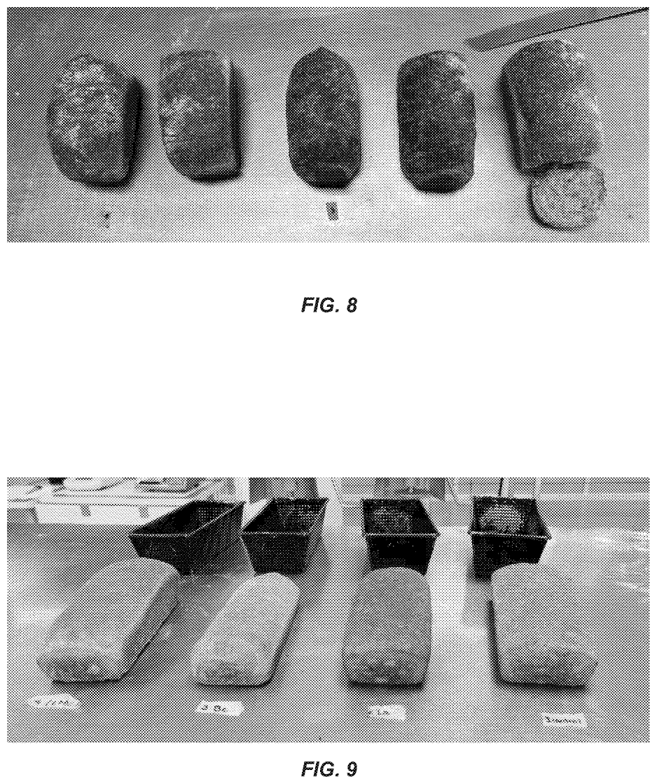

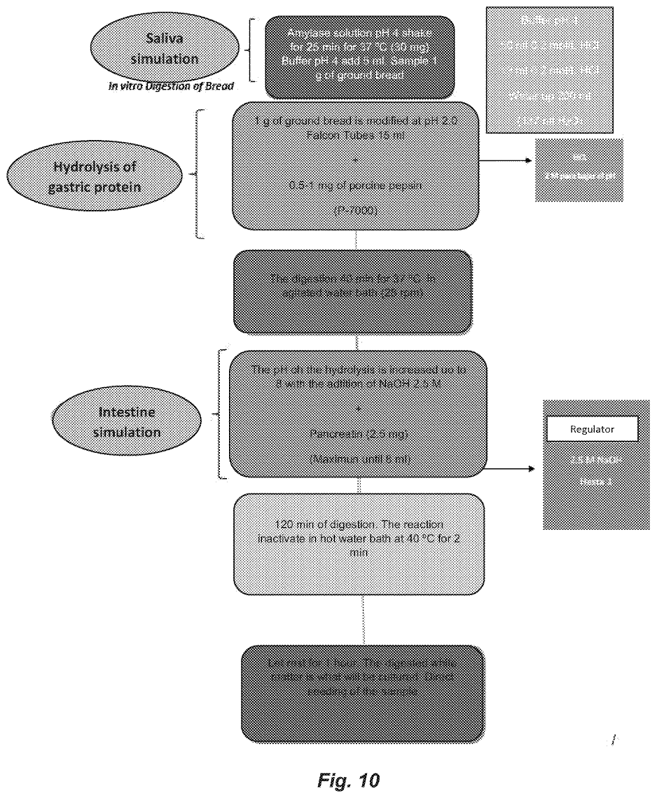

The 3 encapsulated products were tested in triplicate on boxed bread with the recipe provided by the BIMBO® team at a dose of 4 g/100 g of dry products. Breads were obtained with sensory characteristics equal to those of control bread (FIG. 8) and with viability of microorganisms at a concentration higher than 1×10⁶ CFU/g, which is required to say that a food is probiotic (Table 3).

TABLE 3
Results in micro-baking tests at doses of 4 g/100 g

| | | | CFU/Gₚₐₙ |
|---|---|---|---|
| BREAD | CFU/gᵢ | CFU/G_{MASA} | dose 4 g/100 g |
| Control | NA | NA | NA |
| España AB-life | 7.50 × 10¹⁰ | ND | 6.50 × 10²* |
| (positive control) | | | |
| L. acidophilus | 1.22 × 10⁸ | 3.5 × 10⁸ | 1.92 × 10⁶ |
| B. clausii | 3.53 × 10¹⁰ | 1.94 × 10⁸ | 2.58 × 10⁶ |
| 2 probiotics | La. 6.69 × 10⁷ | La. 3.39 × 10⁸ | La. 1.85 × 10⁷ |
| | Bc. 2.24 × 10¹⁰ | Bc. 5.58 × 10⁸ | Bc. 1.58 × 10⁷ |

In encapsulated from Spain, it was not possible to quantify the concentration necessary to be a 1×10⁶ probiotic bread

FIG. 8. Micro-o-breadification prototypes with encapsulations generated by direct method. From left to right you can see the control bread, the bread with microencapsulation from Spain (positive control), encapsulated with L. acidophilus, encapsulated with B. clausii and the encapsulated with the 2 probiotics (L. acidophilus and B. clausii).

The concentration of microencapsulation per 100 g of bread was raised to determine the effective dose. It was concluded that 4 g/100 g to 10 g/100 g is a sufficient dose to obtain cell viability above 1×10⁶ CFU/g of bread as suggested by the Mexican Standards.

Table 4. Results in Micro-Baking Tests at Doses of 10 g/100 g

Commercial-size breads of 460 g were also made, by the sponge method. In these prototypes, differences in the volume of bread were observed. However, these were attributed to the fermentation times used and the percentage of water that was added to the recipe (FIG. 9).

These commercial prototypes, along with enough samples of each

| BREAD | CFU/gᵢ | CFU/G_{MASA} | CFU/Gₚₐₙ |
|---|---|---|---|
| Control | NA | NA | NA |
| España AB-life | 7.50 × 10¹⁰ | 1.22 × 10⁸ | 2.07 × 10⁴ |
| L. acidophilus | 1.22 × 10⁸ | 1.30 × 10⁸ | 6.00 × 10^{2 a 8} |
| B. clausii | 3.53 × 10¹⁰ | 1.31 × 10⁹ | 7.00 × 10^{3 a 8} |
| 2 probiotics | La. 6.69 × 10⁸ | La. 1.23 × 10⁸ | La. 7.37 × 10^{3 a 9} |
| | Bc. 2.24 × 10¹⁰ | Bc. 8.35 × 10⁸ | Bc. 2.55 × 10^{4 a 9} |

microencapsulation to make commercial breads were delivered to BIMBO® so that they can be tested with their bread-making methodology.

pH measurements of the prototype bread were carried out and no changes were observed due to the incorporation of any of the microencapsulates. Likewise, although a greater fermentation is observed in breads added with Lactobacillus acidophilus. It cannot be determined that it is constant, this is precisely attributed to the encapsulation of the microorganism.

With these tests of micro-baking and commercial bread, it is concluded that the highest concentration of microorganisms is found in the central part of the breadcrumb. In addition, that the dose to obtain a concentration greater than 1×10⁶ CFU/g must be at least 4 g/100 g of solid ingredients in the bread, and that up to 10 g/100 g of solid ingredients can be obtained concentrations greater than 1×10⁸ CFU/g in the breadcrumb.

### Stage 3: Life Assessment of Developed Bread Shelf.

It was proposed to test 3 shelf life conditions, considering 3 temperatures and a relative humidity to quantify the shelf life of the final product and the viability of the probiotics in CFU/g.

Temperatures of 37° C., 20° C. and 4° C. were tested at a relative humidity of 60% and it was determined that the shelf life of the product, using the preservatives indicated by BIMBO®, has a shelf life greater than 30 days at 37° C. and increases to 30 days at 20° C. and at 4° C. it has been monitored for at least 30 days with no apparent effect on sensory properties. no fungal growth and viability greater than 1×10⁸ CFU/g. The shelf life is similar in control breads and breads added with probiotics. Annex 1 shows the observation tables for each of the temperatures tested.

### Step 4: In Vitro Digestion Evaluation of the Developed Bread.

It was proposed to evaluate the number of viable microorganisms by performing an in-vitro digestion assay to determine the amount of viable microorganisms that would potentially reach the human intestine.

The samples were digested in triplicate. One gram of each sample was subjected to:
Digestion with porcine amylase at pH 4 for 20 minutes.
Digestion with porcine pepsin at pH 2 for 120 minutes.
Digestion with porcine pancreatin at pH 9.1 for 120 minutes

After digestion, the cell viability of the samples was measured, obtaining the following results shown in Table 5.

In them it is observed that the concentration obtained in the bread was sufficient to be declared as a probiotic bread. Regarding digestion, no additional protection was observed by the encapsulation provided by Spain AB-life compared to the encapsulation obtained at the Tecnológico de Monterrey® to protect a Lactobacillus microorganism.

In the case of the microencapsulates obtained at the Tecnológico de Monterrey®, it is observed that the microorganism that best resisted in-vitro digestion was the sporulated microorganism, although Lactobacillus acidophilus managed to survive at a concentration of 9.0×10¹ CFU per gram.

TABLE 5
Results in microbaking tests at doses of 10 g/100 g with in vitro digestion

| BREAD | CFU/gᵢ | CFU/G_{MASA} | CFU/Gₚₐₙ | CFU/g_{digestion} |
|---|---|---|---|---|
| Control | NA | NA | NA | NA |
| España AB-life | 7.50 × 10¹⁰ | 1.22 × 10⁸ | 2.07 × 10⁴ | 8.5 × 10¹ |
| L. acidophilus | 1.22 × 10⁸ | 1.30 × 10⁸ | 6.00 × 10^{2 a 8} | 9.0 × 10¹ |
| B. clausii | 3.53 × 10¹⁰ | 1.31 × 10⁹ | 7.00 × 10^{3 a 8} | 1.36 × 10⁴ |
| 2 probiotics | La. 6.69 × 10⁸ | La. 1.23 × 10⁸ | La. 7.37 × 10^{3 a 9} | La. Nd |
| | Bc. 2.24 × 10¹⁰ | Bc. 8.35 × 10⁸ | Bc. 2.55 × 10^{4 a 9} | Bc. 1.20 × 10⁴ |

The replication of in-vitro digestion was performed and Table 6 shows the results. As can be seen, the best encapsulation is the double probiotic in a single encapsulation, it is the one that had a better performance not only in baking, but also in the conditions of in-vitro digestion.

TABLE 6
Results in microbaking tests at doses of 10 g/100
g with in-vitro digestion, Replica 1 and 2.

| | Pan | Replica 1 | Replica 2 |
|---|---|---|---|
| 1 | Control | NA | NA |
| 2 | España | 8.5 × 10¹ | 2.5 × 10³ |
| 3 | L. acidophilus | 9.0 × 10¹ | 2.05 × 10² |
| 4 | B. clausii | 1.36 × 10⁴ | 2.95 × 10⁵ |
| 5 | 2 probioticos | La. ND | L.a. 1.52 × 10⁵ |
| | | Bc. 1.20 × 10⁴ | Bc. 2.06 × 10⁵ |

Optimal formulations for microencapsulation were obtained

Basillus clausii with 35% Inulin, 60% pea protein, 5% maltodextrin and an initial concentration of probiotics in the order of 4×10¹¹ CFU.

Lactobacillus acidophilus with 30% Inulin, 40% pea protein, 25% maltodextrin and an initial concentration of probiotics in the order of 4×10⁸ CFU.

Both probiotics with 30% Inulin, 40% pea protein, 25% maltodextrin and an initial concentration of probiotics in the order of 4×10¹¹ CFU for Basillus clausii and 4×10⁸ CFU for Lactobacillus acidophilus.

These formulations could be scaled up to produce 4 g up to 150 g per run in spray drying equipment.

By the size of the microorganisms, it is determined that it is possible to change the microorganism to be encapsulated within the size range of those tested, this by the size of the encapsulated microorganisms that ranged from circular sporulates (less than one micrometer) to bacillion (approximately 2 micrometers) with the formulations obtained.

The optimal dose for the incorporation of bread is between 4 g to 10 g per 100 g of the dry ingredients for making boxed bread, to obtain a viability of between 1×10⁶ and 1×10⁸ from each microorganism.

It is possible to add it to white bread as a wholemeal bread obtaining sufficient viability of the microorganisms.

Boxed bread does not change its sensory properties, nor its shelf life tested at 3 different temperatures.

The formulation that obtained the best performance in the baking process and in vitro digestion according to the viability of probiotics was the double probiotic encapsulation.

### Results in In-Vitro Digestion.

Digestion in-Vito 1 Gram of Sample.
Digestion in-Vito 1 Gram of Sample.

1. An encapsulated probiotic stable at encapsulated baking temperatures for food, the formulation comprising:
probiotic Basillus clausii with 35% Inulin, 60% pea protein, 5% maltodextrin and an initial concentration of probiotics in the order of 4×10¹¹ CFU, and an encapsulation of Basillus clausii 3.53×10¹⁰ CFU/g is obtained at baking temperatures of 170°-210°.

2. The encapsulated probiotic of claim 1, where the encapsulation of the microorganism is determined that it is possible to change the microorganism to be encapsulated within the size range of those tested, this by the size of the encapsulated microorganisms that ranged from circular sporulates of less than one micrometer to bacilli of approximately 2 micrometers.

3. The encapsulated probiotic of claim 1, wherein the formulation is scaled up to produce from 4 g to 150 g per run in spray drying equipment.

4. The encapsulated probiotic of claim 1, where the optimal dose for the incorporation of bread is between 4 g to 10 g per 100 g of the dry ingredients for the production of box bread, to obtain from each microorganism a viability of between 1×10⁶ and 1×10⁸.

5. An encapsulated probiotic stable at encapsulated baking temperatures for food, the formulation comprising:
a Lactobacillus acidophilus probiotic with 30% Inulin, 40% pea protein, 25% maltodextrin and an initial concentration of probiotics in the order of 4×10⁸ CFU resist at baking temperatures of 170°-210°.

6. The encapsulated probiotic of claim 5, where the encapsulation of the microorganism is determined that it is possible to change the microorganism to be encapsulated within the size range of those tested, this by the size of the encapsulated microorganisms that ranged from circular sporulates of less than one micrometer to bacilli of approximately 2 micrometers.

7. The encapsulated probiotic of claim 5, wherein the formulation is scaled up to produce 4 g up to 150 g per run in spray drying equipment.

8. The encapsulated probiotic of claim 5, wherein the optimal dose for the incorporation of bread is between 4 g to 10 g per 100 g of the dry ingredients for the production of box bread, to obtain from each microorganism a viability of between 1×10⁶ and 1×10⁸.

9. The encapsulated probiotic of claim 1, further comprising Lactobacillus acidophilus present at 6.69×10⁸ CFU/g.

10. The encapsulated probiotic of claim 5 further comprising Basillus clausii present at 2.24×10¹⁰ CFU/g.
